(19) United States
(12) Patent Application Publication
(10) Pub. No.: US 2026/0245725 A1
(43) Pub. Date: Aug. 20, 2026

(54) LEFT VENTRICULAR MASS ESTIMATION APPARATUS AND METHOD

(71) Applicants: National Yang Ming Chiao Tung University, Hsinchu City (TW); NATIONAL TAIWAN UNIVERSITY, TAIPEI (TW); National Taiwan University Hospital Hsin-Chu Branch, Hsinchu County (TW); National Taiwan University Hospital, Taipei City (TW)

(72) Inventors: Shin-Mu TSENG, Hsinchu City (TW); Heng-Yu PAN, Hsinchu City (TW); Tzung-Dau WANG, Taipei City (TW); Wen-Jeng LEE, Taipei City (TW)

(21) Appl. No.: 19/056,782

(22) Filed: Feb. 19, 2025

(51) Int. Cl. G16H 50/20; A61B 5/366; G16H 10/60; G16H 40/60; G16H 50/70

(52) U.S. Cl. CPC G16H 50/20; A61B 5/366; G16H 10/60; G16H 40/60; G16H 50/70

(57) ABSTRACT

A left ventricular mass estimation apparatus and method are provided. The apparatus receives an electrocardiogram to be tested, wherein the electrocardiogram to be tested includes multiple lead electrocardiograms corresponding to multiple body surface positions. The apparatus classifies the lead electrocardiograms into groups based on the body surface positions. The apparatus extracts electrocardiogram features from the lead electrocardiograms respectively based on the groups. The apparatus estimates a left ventricular mass corresponding to the electrocardiogram to be tested based on the electrocardiogram features, attribute data corresponding to the electrocardiogram to be tested, and demographic data corresponding to the electrocardiogram to be tested.

### BACKGROUND

### Field of Invention

The present disclosure relates to a left ventricular mass estimation apparatus. More particularly, the present disclosure relates to a left ventricular mass estimation apparatus based on electrocardiograms.

### Description of Related Art

One of the characteristics of left ventricular hypertrophy (LVH) symptoms is the increase of left ventricular myocardial mass (LVM), which may lead to cardiovascular disease. Conventional diagnosis of left ventricular hypertrophy utilizes limited electrocardiogram features and is based on fixed criteria, thus the diagnosis lacks sensitivity.

On the other hand, accurate estimation of left ventricular myocardial mass requires imaging techniques such as magnetic resonance imaging or computed tomography, and provides prognostic information beyond the symptoms of left ventricular hypertrophy. However, this method is costly and time-consuming.

In view of this, how to provide a convenient and accurate left ventricular mass estimation technology is the goal that the industry strives to work on.

### SUMMARY

The disclosure provides a left ventricular mass estimation apparatus comprising a communication interface and a processor. The communication interface is configured to receive an electrocardiogram to be tested, wherein the electrocardiogram to be tested comprises a plurality of lead electrocardiograms corresponding to a plurality of body surface positions. The processor is electrically connected to the communication interface and configured to execute the following operations: classifying the lead electrocardiograms into a plurality of groups based on the body surface positions; extracting a plurality of electrocardiogram features from the lead electrocardiograms respectively based on the groups; and estimating a left ventricular mass corresponding to the electrocardiogram to be tested based on the electrocardiogram features, attribute data corresponding to the electrocardiogram to be tested, and demographic data corresponding to the electrocardiogram to be tested.

The disclosure further provides a left ventricular mass estimation method, being adapted for use in an electronic apparatus, wherein the left ventricular mass estimation method comprises the following steps: obtaining an electrocardiogram to be tested, wherein the electrocardiogram to be tested comprises a plurality of lead electrocardiograms corresponding to a plurality of body surface positions; classifying the lead electrocardiograms into a plurality of groups based on the body surface positions; extracting a plurality of electrocardiogram features from the lead electrocardiograms respectively based on the groups; and estimating a left ventricular mass corresponding to the electrocardiogram to be tested based on the electrocardiogram features, attribute data corresponding to the electrocardiogram to be tested, and demographic data corresponding to the electrocardiogram to be tested.

It is to be understood that both the foregoing general description and the following detailed description are by examples, and are intended to provide further explanation of the disclosure as claimed.

### BRIEF DESCRIPTION OF THE DRAWINGS

The disclosure can be more fully understood by reading the following detailed description of the embodiment, with reference made to the accompanying drawings as follows:

FIG. 1 is a schematic diagram illustrating a left ventricular mass estimation apparatus according to a first embodiment of the present disclosure.

FIG. 2 is a schematic diagram illustrating operations of the left ventricular mass estimation apparatus according to some embodiments of the present disclosure.

FIG. 3 is a flow diagram illustrating a left ventricular mass estimation method according to a second embodiment of the present disclosure.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts.

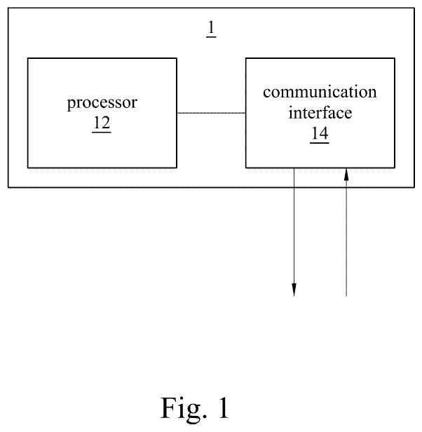

Please refer to FIG. 1, which is a schematic diagram illustrating a left ventricular mass estimation apparatus 1 according to a first embodiment of the present disclosure. The left ventricular mass estimation apparatus 1 comprises a processor 12 and a communication interface 14, wherein the processor 12 is electrically connected to the communication interface 14. The left ventricular mass estimation apparatus 1 is configured to estimate a left ventricular mass of a patient.

The communication interface 14 is configured to receive an electrocardiogram to be tested of the patient. In some embodiments, the left ventricular mass estimation apparatus 1 is connected to equipment with electrocardiogram measurement function such as electrocardiographs or wearable devices via the communication interface 14, so that the left ventricular mass estimation apparatus 1 may receive the electrocardiogram to be tested from the equipment. In some embodiments, the communication interface 14 may comprises wired and/or wireless interface such as Wi-Fi, Bluetooth, and/or Ethernet interface.

The electrocardiogram to be tested received by the communication interface 14 comprises a plurality of lead electrocardiograms corresponding to a plurality of body surface positions. For example, the electrocardiogram to be tested received by the communication interface 14 comprises 12—lead electrocardiograms (i.e., the electrocardiograms measured from 12 positions on human body surface), wherein the 12 leads comprise I, II, III, aVR, aVL, aVF, V1, V2, V3, V4, V5, and V6. In some embodiments, the lead electrocardiograms comprise electrocardiograms measured from multiple body surface positions in the same time period. In some embodiments, the lead electrocardiograms comprise electrocardiograms measured in a period of time (e.g., 10 seconds).

In some embodiments, the processor 12 comprises a central processing unit (CPU), a graphics processing unit (GPU), a microcontroller unit (MCU), a multi-processor, a distributed processing system, an application specific integrated circuit (ASIC), and/or a suitable processing unit.

First, in order to analyze signal features of the electrocardiogram, the left ventricular mass estimation apparatus 1 classifies the lead electrocardiograms according to the measured position of each of the lead electrocardiograms.

Next, the left ventricular mass estimation apparatus 1 extracts electrocardiogram features from the different groups of lead electrocardiograms respectively based on classification result.

After extracting the electrocardiogram features, the left ventricular mass estimation apparatus 1 estimates a left ventricular mass of the patient by combining the electrocardiogram features, and attribute data and demographic data corresponding to the electrocardiogram to be tested.

Specifically, the processor 12 is configured to execute the following operations: classifying the lead electrocardiograms into a plurality of groups based on the body surface positions; extracting a plurality of electrocardiogram features from the lead electrocardiograms respectively based on the groups; and estimating a left ventricular mass corresponding to the electrocardiogram to be tested based on the electrocardiogram features, attribute data corresponding to the electrocardiogram to be tested, and demographic data corresponding to the electrocardiogram to be tested.

The electrocardiograms measured from different positions will present different signal features on the same depolarization vector. Therefore, through the above operations, the left ventricular mass estimation apparatus 1 is able to extract features for different measured positions respectively and perform the estimation accordingly to increase the accuracy of the estimation. On the other hand, by combining the extracted electrocardiogram features, the demographic data of the patient, and the attribute data of the electrocardiogram to be tested, the left ventricular mass estimation apparatus 1 may also estimate the left ventricular mass based on other information of the electrocardiogram and the physiological condition of the patient.

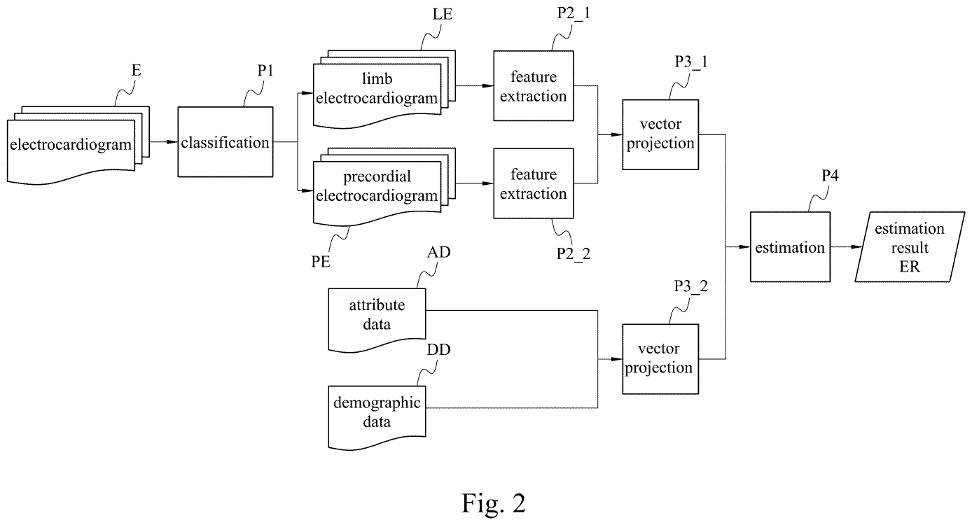

In order to describe the present invention more specifically, please refer to FIG. 2, which is a schematic diagram illustrating operations of the left ventricular mass estimation apparatus 1 according to some embodiments of the present disclosure.

First, in an operation P1, the left ventricular mass estimation apparatus 1 classifies multiple electrocardiograms E into lead electrocardiograms LE belonging to a limb group and precordial electrocardiograms PE belonging to a precordial group, wherein the electrocardiograms E comprise multiple electrocardiograms corresponding to multiple body surface positions (i.e., the aforementioned lead electrocardiograms) measured from a patient in the same time period.

Specifically, the operation of the processor 12 classifying the lead electrocardiograms into the groups further comprises: classifying the lead electrocardiograms into a limb group and a precordial group, wherein the limb group comprises the lead electrocardiograms measured from human limbs, and the precordial group comprises the lead electrocardiograms measured from human chest.

For example, the electrocardiograms E comprise 12 leads of electrocardiogram, and the left ventricular mass estimation apparatus 1 classifies the electrocardiograms of I, II, III, aVR, aVL, and aVF leads into the limb group (i.e., the limb electrocardiograms LE) and classifies the electrocardiograms of V1, V2, V3, V4, V5, and V6 leads into the precordial group (i.e., the precordial electrocardiograms PE).

It is noted that, since the measured positions of the limb electrocardiograms LE and the precordial electrocardiograms PE are different, the observation angles of the depolarization vector are also different. Therefore, the left ventricular mass estimation apparatus 1 may utilize different calculation approaches on the different groups.

After completing the classification, in operations P2_1 and P2_2, the left ventricular mass estimation apparatus 1 extracts electrocardiogram features of the limb electrocardiograms LE and the precordial electrocardiograms PE respectively.

Specifically, the processor 12 extracts electrocardiogram features of the limb electrocardiograms LE and the precordial electrocardiograms PE respectively by using a feature extraction layer to generate a feature vector. In some embodiments, the feature extraction layer utilized in the operation P2_1 is different from the feature extraction layer utilized in the operation P2_2, and the left ventricular mass estimation apparatus 1 may perform feature extraction by using different approaches on different types of electrocardiograms. In some embodiments, the operation P2_1 and P2_2 each generate a 10*1 size feature vector.

In some embodiments, before extracting the electrocardiogram features, the left ventricular mass estimation apparatus 1 segments electrocardiograms of a single heartbeat from the electrocardiograms E and then performs the feature extraction on the heartbeat. Since each heartbeats in a short period of time should present similar features, the left ventricular mass estimation apparatus 1 may perform analysis on electrocardiograms of one of the heartbeats and avoid being biased by the heart rate and minor difference between multiple heartbeats.

Specifically, the operation of the processor 12 extracting the electrocardiogram features further comprises: segmenting a plurality of heartbeat electrocardiograms corresponding to a heartbeat from the lead electrocardiograms based on a waveform of one of the lead electrocardiograms; and extracting the electrocardiogram features from the heartbeat electrocardiograms respectively based on the groups.

For example, assumed that the electrocardiograms E record 10—second signals of multiple heartbeats. In accordance, the left ventricular mass estimation apparatus 1 ignores the first and the last heartbeats and segments the signals of one of the other heartbeats to ensure that the segmented signals record the whole heartbeat. More specifically, since the R wave is the most significant signal in an electrocardiogram, the left ventricular mass estimation apparatus 1 takes the R wave signal as reference and segments the signal in a time period before and after the R wave as a single-heartbeat electrocardiogram, e.g., the signal from 400 milliseconds before the R wave to 600 milliseconds after the R wave. In some embodiments, the left ventricular mass estimation apparatus 1 segments the heartbeat electrocardiograms in the same time period from multiple leads of electrocardiogram to capture the signals of the same heartbeat.

In some embodiments, since electrocardiograms are time-related data, the left ventricular mass estimation apparatus 1 performs feature extraction on timing attributes of the electrocardiograms.

Specifically, the operation of the processor 12 extracting the electrocardiogram features further comprises: extracting the electrocardiogram features from the lead electrocardiograms based on a plurality of timing attributes of a plurality of signals in the lead electrocardiograms.

For example, the left ventricular mass estimation apparatus 1 performs feature extraction on the limb electrocardiograms LE and the precordial electrocardiograms PE respectively by utilizing the feature extraction layer of a Temporal Convolutional Network (TCN).

In the technical field of left ventricular mass estimation, the electrocardiograms corresponding to different patient genders will present different features. Therefore, in some embodiments, the left ventricular mass estimation apparatus 1 utilizes different feature extraction layers for different patient genders to extract different aspects of the electrocardiogram features.

Specifically, the operation of the processor 12 extracting the electrocardiogram features further comprises: selecting one of a plurality of feature extraction layers based on gender data of the demographic data; and extracting the electrocardiogram features from the lead electrocardiograms based on the selected one of the feature extraction layers.

For example, T wave segments in electrocardiograms of biological males in 3 groups of low, medium, and high left ventricular mass show significant differences. Accordingly, the feature extraction layer corresponding to male will focus on the feature extraction on T wave segments. On the other hand, in electrocardiograms of biological females in different groups of left ventricular mass, QRS wave segments show significant differences instead of T wave segments. In accordance, the feature extraction layer corresponding to female will focus on the feature extraction on QRS wave segments.

Next, in an operation P3_1, the left ventricular mass estimation apparatus 1 projects the electrocardiogram features extracted in operations P2_1 and P2_2 onto vectors of the same dimension.

Specifically, the operation of the processor 12 estimating the left ventricular mass further comprises: projecting the electrocardiogram features onto a first vector based on a first projection layer; and estimating the left ventricular mass corresponding to the electrocardiogram to be tested based on the first vector, the attribute data, and the demographic data.

For example, the left ventricular mass estimation apparatus 1 projects two feature vectors (i.e., the electrocardiogram features) onto the same vector by using a projection layer of Multilayer Perceptron (MLP). In some embodiments, the left ventricular mass estimation apparatus 1 projects two 10*1 size feature vectors onto a 1024*1 size vector.

On the other hand, in an operation P3_2, the left ventricular mass estimation apparatus 1 projects the attribute data AD of the electrocardiograms E and the demographic data DD of the patient onto a vector.

The attribute data AD indicates the information of the electrocardiograms E other than the signal, comprising P-R-T axes (i.e., the electrical axis of heart) and/or QRS wave duration (i.e., the time length of the segment comprising Q wave, R wave, and S wave in an electrocardiogram). The demographic data DD indicates the physiological data of the patient, e.g., age, gender, height, and/or weight.

Specifically, the operation of the processor 12 estimating the left ventricular mass further comprises: projecting the attribute data and the demographic data onto a second vector based on a second projection layer; and estimating the left ventricular mass corresponding to the electrocardiogram to be tested based on the second vector and the electrocardiogram features.

For example, after combining 4 columns of data in the attribute data AD comprising QRS wave duration and P axis, R axis, and T axis of P-R-T axes and 4 columns of data in the demographic data DD comprising age, gender, height, and weight, the left ventricular mass estimation apparatus 1 projects the 8 columns of data onto the same vector (e.g., a 8*1 size of vector). In some embodiments, the operation P3_1 and/or P3_2 comprises embedding operations of a machine learning model.

Finally, in an operation P4, the left ventricular mass estimation apparatus 1 estimates the left ventricular mass and generates an estimation result ER based on the vectors generated in the operations P3_1 and P3_2.

For example, the left ventricular mass estimation apparatus 1 generates the estimation result ER comprising the left ventricular mass based on the vectors by using a prediction layer of Multilayer Perceptron.

Similarly, in some embodiments, since different feature extraction approaches are utilized for different genders, the left ventricular mass estimation apparatus 1 utilizes different prediction approaches for different genders in the operation P3_1.

Specifically, the operation of the processor 12 estimating the left ventricular mass further comprises: selecting one of a plurality of prediction layers based on gender data of the demographic data; and generating a prediction result by using the selected one of the prediction layers based on the electrocardiogram features, the attribute data, and the demographic data, wherein the prediction result comprises the left ventricular mass.

In some embodiments, the aforementioned feature extraction layer, projection layer, and/or prediction layer are generated after training by multiple training data, wherein the training data comprises electrocardiograms, attribute data, demographic data, and the corresponding left ventricular mass. Accordingly, the trained feature extraction layer, projection layer, and/or prediction layer are able to estimate the left ventricular mass of the patient based on the electrocardiogram to be tested.

Specifically, the processor 12 extracts the electrocardiogram features from the lead electrocardiograms based on a feature extraction layer; the processor 12 estimates the left ventricular mass corresponding to the electrocardiogram to be tested based on a prediction layer; and the feature extraction layer and the prediction layer are generated through the following operations: generating a prediction result by using an initial feature extraction layer and an initial prediction layer based on a plurality of training electrocardiograms; adjusting a plurality of parameters of the initial feature extraction layer and the initial prediction layer based on the prediction result and a plurality of known left ventricular masses corresponding to the training electrocardiograms; and taking the adjusted initial feature extraction layer and the adjusted initial prediction layer as the feature extraction layer and the prediction layer.

It is noted that, the training approach for each layers (e.g., the feature extraction layer, projection layer, and/or prediction layer) may be adjusted according to the operation of the left ventricular mass estimation apparatus 1. For example, in the embodiment of performing calculations by using different layers for different patient genders, layers corresponding to different genders will be trained by using training data of different genders in the training phase. Accordingly, the left ventricular mass estimation apparatus 1 is able to select the corresponding layer to perform estimation.

In summary, the left ventricular mass estimation apparatus 1 provided by the present disclosure is able to extract features from the electrocardiograms measured from different body surface positions and estimate the left ventricular mass by combining the attribute data of electrocardiograms and the demographic data of the patient. Additionally, the left ventricular mass estimation apparatus 1 may utilizes different calculation approaches for different patient genders to increase the estimation accuracy. As a result, the left ventricular mass estimation apparatus 1 does not require complex examination to provide a flexible and accurate left ventricular mass estimation technology.

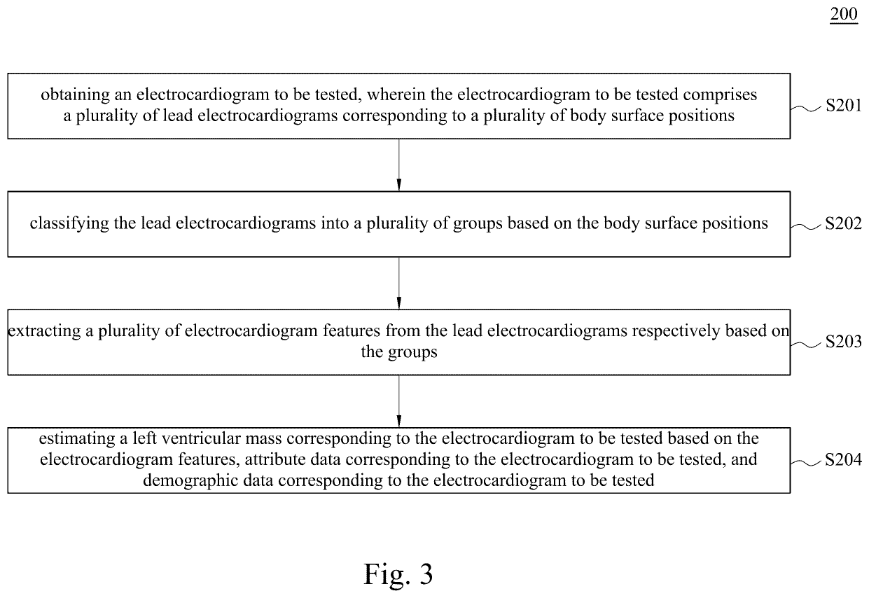

Please refer to FIG. 3, which is a flow diagram illustrating a left ventricular mass estimation method 200 according to a second embodiment of the present disclosure. The left ventricular mass estimation method 200 comprises steps S201-S204. The left ventricular mass estimation method 200 is configured to estimate a left ventricular mass of a patient. The left ventricular mass estimation method 200 can be executed by an electronic apparatus (e.g., the left ventricular mass estimation apparatus 1 in the first embodiment).

First, in the step S201, the electronic apparatus obtains an electrocardiogram to be tested, wherein the electrocardiogram to be tested comprises a plurality of lead electrocardiograms corresponding to a plurality of body surface positions.

Next, in the step S202, the electronic apparatus classifies the lead electrocardiograms into a plurality of groups based on the body surface positions.

Next, in the step S203, the electronic apparatus extracts a plurality of electrocardiogram features from the lead electrocardiograms respectively based on the groups.

Finally, in the step S204, the electronic apparatus estimates a left ventricular mass corresponding to the electrocardiogram to be tested based on the electrocardiogram features, attribute data corresponding to the electrocardiogram to be tested, and demographic data corresponding to the electrocardiogram to be tested.

In some embodiments, the step S202 further comprises the electronic apparatus classifying the lead electrocardiograms into a limb group and a precordial group, wherein the limb group comprises the lead electrocardiograms measured from human limbs, and the precordial group comprises the lead electrocardiograms measured from human chest.

In some embodiments, the step S203 further comprises the electronic apparatus segmenting a plurality of heartbeat electrocardiograms corresponding to a heartbeat from the lead electrocardiograms based on a waveform of one of the lead electrocardiograms; and the electronic apparatus extracting the electrocardiogram features from the heartbeat electrocardiograms respectively based on the groups.

In some embodiments, the step S203 further comprises the electronic apparatus extracting the electrocardiogram features from the lead electrocardiograms based on a plurality of timing attributes of a plurality of signals in the lead electrocardiograms.

In some embodiments, the step S203 further comprises the electronic apparatus selecting one of a plurality of feature extraction layers based on gender data of the demographic data; and the electronic apparatus extracting the electrocardiogram features from the lead electrocardiograms based on the selected one of the feature extraction layers.

In some embodiments, the step S204 further comprises the electronic apparatus projecting the electrocardiogram features onto a first vector based on a first projection layer; and the electronic apparatus estimating the left ventricular mass corresponding to the electrocardiogram to be tested based on the first vector, the attribute data, and the demographic data.

In some embodiments, the step S204 further comprises the electronic apparatus projecting the attribute data and the demographic data onto a second vector based on a second projection layer; and the electronic apparatus estimating the left ventricular mass corresponding to the electrocardiogram to be tested based on the second vector and the electrocardiogram features.

In some embodiments, the step S204 further comprises the electronic apparatus selecting one of a plurality of prediction layers based on gender data of the demographic data; and the electronic apparatus generating a prediction result by using the selected one of the prediction layers based on the electrocardiogram features, the attribute data, and the demographic data, wherein the prediction result comprises the left ventricular mass.

In some embodiments, the left ventricular mass estimation method extracts the electrocardiogram features from the lead electrocardiograms based on a feature extraction layer; the left ventricular mass estimation method estimates the left ventricular mass corresponding to the electrocardiogram to be tested based on a prediction layer; and the feature extraction layer and the prediction layer are generated through the following steps: generating a prediction result by using an initial feature extraction layer and an initial prediction layer based on a plurality of training electrocardiograms; adjusting a plurality of parameters of the initial feature extraction layer and the initial prediction layer based on the prediction result and a plurality of known left ventricular masses corresponding to the training electrocardiograms; and taking the adjusted initial feature extraction layer and the adjusted initial prediction layer as the feature extraction layer and the prediction layer.

In some embodiments, the attribute data comprises a QRS wave duration and an electrical axis corresponding to the electrocardiogram to be tested.

In some embodiments, the demographic data comprises height, weight, age, gender of a patient corresponding to the electrocardiogram to be tested.

In summary, the left ventricular mass estimation method 200 provided by the present disclosure is able to extract features from the electrocardiograms measured from different body surface positions and estimate the left ventricular mass by combining the attribute data of electrocardiograms and the demographic data of the patient. Additionally, the left ventricular mass estimation method 200 may utilizes different calculation approaches for different patient genders to increase the estimation accuracy. As a result, the left ventricular mass estimation method 200 does not require complex examination to provide a flexible and accurate left ventricular mass estimation technology.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

### EXPLANATION OF THE SYMBOLS IN THE FIGURES

1: left ventricular mass estimation apparatus
12: processor
14: communication interface
E: electrocardiograms
LE: limb electrocardiograms
PE: precordial electrocardiograms
AD: attribute data
DD: demographic data
ER: estimation result
P1,P2_1,P2_2,P3_1,P3_2,P4: operations
200: left ventricular mass estimation method
S201~S204: Steps

### BIOLOGICAL MATERIAL STORAGE

Domestic storage information (please note the order of storage institution, date and number)
N/A
Overseas deposit information (please note in the order of deposit country, institution, date and number)
N/A

What is claimed is:

1. A left ventricular mass estimation apparatus, comprising:
a communication interface, configured to receive an electrocardiogram to be tested, wherein the electrocardiogram to be tested comprises a plurality of lead electrocardiograms corresponding to a plurality of body surface positions; and
a processor, electrically connected to the communication interface, configured to execute the following operations:
classifying the lead electrocardiograms into a plurality of groups based on the body surface positions;
extracting a plurality of electrocardiogram features from the lead electrocardiograms respectively based on the groups; and
estimating a left ventricular mass corresponding to the electrocardiogram to be tested based on the electrocardiogram features, attribute data corresponding to the electrocardiogram to be tested, and demographic data corresponding to the electrocardiogram to be tested.

2. The left ventricular mass estimation apparatus of claim 1, wherein the operation of classifying the lead electrocardiograms into the groups further comprises:
classifying the lead electrocardiograms into a limb group and a precordial group, wherein the limb group comprises the lead electrocardiograms measured from human limbs, and the precordial group comprises the lead electrocardiograms measured from human chest.

3. The left ventricular mass estimation apparatus of claim 1, wherein the operation of extracting the electrocardiogram features further comprises:
segmenting a plurality of heartbeat electrocardiograms corresponding to a heartbeat from the lead electrocardiograms based on a waveform of one of the lead electrocardiograms; and
extracting the electrocardiogram features from the heartbeat electrocardiograms respectively based on the groups.

4. The left ventricular mass estimation apparatus of claim 1, wherein the operation of extracting the electrocardiogram features further comprises:
extracting the electrocardiogram features from the lead electrocardiograms based on a plurality of timing attributes of a plurality of signals in the lead electrocardiograms.

5. The left ventricular mass estimation apparatus of claim 1, wherein the operation of extracting the electrocardiogram features further comprises:
selecting one of a plurality of feature extraction layers based on gender data of the demographic data; and
extracting the electrocardiogram features from the lead electrocardiograms based on the selected one of the feature extraction layers.

6. The left ventricular mass estimation apparatus of claim 1, wherein the operation of estimating the left ventricular mass further comprises:
projecting the electrocardiogram features onto a first vector based on a first projection layer; and
estimating the left ventricular mass corresponding to the electrocardiogram to be tested based on the first vector, the attribute data, and the demographic data.

7. The left ventricular mass estimation apparatus of claim 1, wherein the operation of estimating the left ventricular mass further comprises:
projecting the attribute data and the demographic data onto a second vector based on a second projection layer; and
estimating the left ventricular mass corresponding to the electrocardiogram to be tested based on the second vector and the electrocardiogram features.

8. The left ventricular mass estimation apparatus of claim 1, wherein the operation of estimating the left ventricular mass further comprises:
selecting one of a plurality of prediction layers based on gender data of the demographic data; and
generating a prediction result by using the selected one of the prediction layers based on the electrocardiogram features, the attribute data, and the demographic data, wherein the prediction result comprises the left ventricular mass.

9. The left ventricular mass estimation apparatus of claim 1, wherein:
the processor extracts the electrocardiogram features from the lead electrocardiograms based on a feature extraction layer;
the processor estimates the left ventricular mass corresponding to the electrocardiogram to be tested based on a prediction layer; and
the feature extraction layer and the prediction layer are generated through the following operations:
generating a prediction result by using an initial feature extraction layer and an initial prediction layer based on a plurality of training electrocardiograms;
adjusting a plurality of parameters of the initial feature extraction layer and the initial prediction layer based on the prediction result and a plurality of known left ventricular masses corresponding to the training electrocardiograms; and
taking the adjusted initial feature extraction layer and the adjusted initial prediction layer as the feature extraction layer and the prediction layer.

10. The left ventricular mass estimation apparatus of claim 1, wherein the attribute data comprises a QRS wave duration and an electrical axis corresponding to the electrocardiogram to be tested.

11. A left ventricular mass estimation method, being adapted for use in an electronic apparatus, wherein the left ventricular mass estimation method comprises the following steps:
obtaining an electrocardiogram to be tested, wherein the electrocardiogram to be tested comprises a plurality of lead electrocardiograms corresponding to a plurality of body surface positions;
classifying the lead electrocardiograms into a plurality of groups based on the body surface positions;
extracting a plurality of electrocardiogram features from the lead electrocardiograms respectively based on the groups; and
estimating a left ventricular mass corresponding to the electrocardiogram to be tested based on the electrocardiogram features, attribute data corresponding to the electrocardiogram to be tested, and demographic data corresponding to the electrocardiogram to be tested.

12. The left ventricular mass estimation method of claim 11, wherein the step of classifying the lead electrocardiograms into the groups further comprises:
classifying the lead electrocardiograms into a limb group and a precordial group, wherein the limb group comprises the lead electrocardiograms measured from human limbs, and the precordial group comprises the lead electrocardiograms measured from human chest.

13. The left ventricular mass estimation method of claim 11, wherein the step of extracting the electrocardiogram features further comprises:
segmenting a plurality of heartbeat electrocardiograms corresponding to a heartbeat from the lead electrocardiograms based on a waveform of one of the lead electrocardiograms; and
extracting the electrocardiogram features from the heartbeat electrocardiograms respectively based on the groups.

14. The left ventricular mass estimation method of claim 11, wherein the step of extracting the electrocardiogram features further comprises:
extracting the electrocardiogram features from the lead electrocardiograms based on a plurality of timing attributes of a plurality of signals in the lead electrocardiograms.

15. The left ventricular mass estimation method of claim 11, wherein the step of extracting the electrocardiogram features further comprises:
selecting one of a plurality of feature extraction layers based on gender data of the demographic data; and
extracting the electrocardiogram features from the lead electrocardiograms based on the selected one of the feature extraction layers.

16. The left ventricular mass estimation method of claim 11, wherein the step of estimating the left ventricular mass further comprises:
projecting the electrocardiogram features onto a first vector based on a first projection layer; and
estimating the left ventricular mass corresponding to the electrocardiogram to be tested based on the first vector, the attribute data, and the demographic data.

17. The left ventricular mass estimation method of claim 11, wherein the step of estimating the left ventricular mass further comprises:
projecting the attribute data and the demographic data onto a second vector based on a second projection layer; and
estimating the left ventricular mass corresponding to the electrocardiogram to be tested based on the second vector and the electrocardiogram features.

18. The left ventricular mass estimation method of claim 11, wherein the step of estimating the left ventricular mass further comprises:
selecting one of a plurality of prediction layers based on gender data of the demographic data; and
generating a prediction result by using the selected one of the prediction layers based on the electrocardiogram features, the attribute data, and the demographic data, wherein the prediction result comprises the left ventricular mass.

19. The left ventricular mass estimation method of claim 11, wherein:
the left ventricular mass estimation method extracts the electrocardiogram features from the lead electrocardiograms based on a feature extraction layer;
the left ventricular mass estimation method estimates the left ventricular mass corresponding to the electrocardiogram to be tested based on a prediction layer; and
the feature extraction layer and the prediction layer are generated through the following steps:
generating a prediction result by using an initial feature extraction layer and an initial prediction layer based on a plurality of training electrocardiograms;
adjusting a plurality of parameters of the initial feature extraction layer and the initial prediction layer based on the prediction result and a plurality of known left ventricular masses corresponding to the training electrocardiograms; and
taking the adjusted initial feature extraction layer and the adjusted initial prediction layer as the feature extraction layer and the prediction layer.

20. The left ventricular mass estimation method of claim 11, wherein the attribute data comprises a QRS wave duration and an electrical axis corresponding to the electrocardiogram to be tested.
